(19) United States
(12) Patent Application Publication
(10) Pub. No.: US 2005/0059831 A1
(43) Pub. Date: Mar. 17, 2005

(54) Preparation of substituted butenolides via palladium-free etherification and amination of masked mucohalic acids

(76) Inventors: Peter Garth Blazecka, Windsor (CA); Ji Zhang, Ann Arbor, MI (US)

Correspondence Address: WARNER-LAMBERT COMPANY, 2800 PLYMOUTH RD, ANN ARBOR, MI, 48105, US

(21) Appl. No.: 10/942,557

(22) Filed: Sep. 16, 2004

Related U.S. Application Data
(60) Provisional application No. 60/503,764, filed on Sep. 17, 2003.

(51) Int. Cl. C07D 307/32

(52) U.S. Cl. 549/313

(57) ABSTRACT

Methods and materials for preparing 4-substituted-2-buten-4-olides are disclosed. The methods include reacting a masked mucohalic acid with a primary or secondary amine or with an arylol in the presence of a base. Unlike existing processes, the disclosed methods do not require the use of palladium, which make them well suited for preparing intermediates in drug syntheses.

### BACKGROUND OF THE INVENTION

1. Field of the Invention

This invention relates to materials and methods for preparing substituted butenolides, which are useful intermediates for preparing biologically active natural products and compounds, including polyketide mycotoxins and nucleosides having anti-viral or anti-bacterial activity.

2. Discussion

Substituted butenolides are useful building blocks for preparing biologically active natural products and synthetic compounds. Useful substituted butenolides include 4-aryloxy-2-buten-4-olides, which may be used as starting materials and intermediates for preparing polyketide mycotoxins, including afflatoxin B₁ and B₂a (B. M. Trost & F. D. Toste, J. Am. Chem. Soc. (2003) 125:3090). Other useful substituted butenolides include 4-amino-2-buten-4-olides, which may be used to prepare nucleosides having known anti-bacterial or anti-viral properties, including Showdomycin, Noraristermycin, Ribavirin, Zidovudine (AZT), Zalcitabine (dideoxycytidine), Bromovinyldeoxyuridine (BVDU), and Abacavir (see, e.g., Agrofoglio et al., Chem. Rev. (2003) 103:1875; L. S. Hegedus et al., J. Org. Chem. (2002) 67:4076; B. M. Trost & Z. Shi, J. Am. Chem. Soc. (1996) 118:3037; U.S. Pat. No. 5,665,721; U.S. Pat. No. 5,034,394).

A potential method for preparing 4-substituted-2-buten-4-olides is palladium-catalyzed allylic alkylation of masked mucohalic acids, which are represented by compounds of Formula 1, 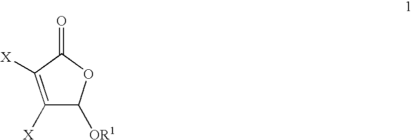
in which X is halogen (Cl, Br, or I), and OR¹ is a leaving group, such as C₁₋₆ alkoxycarbonyloxy or C₁₋₆ alkanoyloxy. As described below, compounds of Formula 1 can be prepared from commercially available mucohalic acids, including mucochloric acid (3,4-dichloro-5-hydroxy-5H-furan-2-one) and mucobromic acid (3,4-dibromo-5-hydroxy-5H-furan 2-one). Though chemists have largely ignored mucohalic acids because of perceived problems associated with regioselectivity and stability under basic conditions, recent investigations have revealed mucohalic acids to be versatile and cost-effective building blocks in drug discovery and development (J. Zhang et al., Tetrahedron Lett. (2003) 44:5579; J. Zhang et al., Org. Lett. (2003) 5:553; J. Zhang et al., Org. Lett. (2002) 4:4559).

The use of masked mucohalic acids to prepare 4-substituted-2-buten-4-olides would appear to be problematic because of the presence of vinyl halide and allylic functional groups. Indeed, the research literature provides few examples of selective manipulation of allylic and vinylic functional groups within the same molecule, and these examples involve using palladium catalysis to differentiate between the two groups (M. G. Organ et al., Tetrahedron Lett. (2003) 44:4403; M. G. Organ et al. J. Am. Chem. Soc. (2002) 124:1288; B. M. Trost & J. D. Oslob J. Am. Chem. Soc. (1999) 121:3057; G. C. Nwokogu J. Org. Chem. (1985) 50:3900). Furthermore, the vinyl halide group is reactive towards a variety of nucleophilic reagents, and undergoes a Michael-Addition-Elimination process in their presence (G. A. Sulikowski et al. J. Org. Chem. (2000) 65:337; H. W. Moore et al. J. Am. Chem. Soc. (1981) 103:1769).

A plausible way to overcome these difficulties would be to activate the 4-position (i.e., γ-position) of the masked mucohalic acid via the formation of a π-allyl-Pd complex that is subsequently attacked by a nucleophile (e.g., reactant having an aryloxy or amino functional group). This technique—palladium-catalyzed allylic alkylation or Tsuji-Trost reaction—has been the subject of numerous investigations (J. Tsuji et al. Tetrahedron Lett. (1965) 5:4387; B. M. Trost & C. Lee, in Catalytic Asymmetric Synthesis, (edited by I. Ojima, 2d ed. 2000) 593-649; J. Tsuji & I. Minami, Acc. Chem. Res. (1987) 20:140; B. M. Trost, Acc. Chem. Res. (1980) 13:385; B. M. Trost, Tetrahedron (1997) 33:2615). The Tsuji-Trost reaction is an important technique in catalytic asymmetric (enantioselective) synthesis (B. M. Trost & D. L. Van Vranken, Chem. Rev. (1996) 96:395) and represents an efficient and powerful method of forming carbon-carbon bonds (J. Tsuji et al., Tetrahedron Lett. (1982) 23:5189; B. M. Trost & T. R. Verhoeven, J. Org. Chem. (1976) 41:3215), carbon-oxygen bonds (B. M. Trost et al., J. Am. Chem. Soc. (2003) 125:9276), carbon-nitrogen bonds (B. M. Trost & D. E. Patterson, (1999) Chem. Eur. J. 5:3279; B. M. Trost et al., J. Am. Chem. Soc. (1996) 118:6297), and carbon-sulfur bonds (M. Frank & H.-J. Gais, Tetrahedron: Asymmetry (1998) 9:3353; B. M. Trost et al., J. Am. Chem. Soc. (1995) 117:9662; C. Goux et al., Tetrahedron Lett. (1992) 33:8099; B. M. Trost & T. S. Scanlan, Tetrahedron Lett. (1986) 27:4141).

A drawback of the Tsuji-Trost methodology is its reliance on palladium. Though used in small quantities, palladium reagents are expensive, which adds to the cost of products made using them. Moreover, FDA regulations place stringent requirements on the permissible level of palladium in drug products (currently 10-50 ppm). Any processes for preparing drug substances that employ palladium reagents should include provisions for removing Pd at some step of the preparative method, which may add to the complexity of the process and to the cost of goods. Therefore, what is needed is a palladium-free method for preparing 4-substituted-2-buten-4-olides from compounds of Formula 1.

### SUMMARY OF THE INVENTION

The present invention provides a palladium-free method of making 4-substituted-2-buten-4-olides. The method should result in cost savings over existing processes that use palladium catalysts, and is particularly advantageous for preparing pharmaceutical products, which have stringent limitations on the permissible level of palladium in the final drug product.

One aspect of the present invention provides a method for preparing compounds represented by Formula 2, 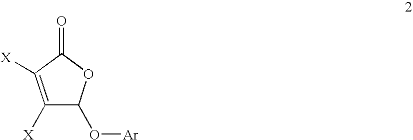
in which X is halogen (typically Cl or Br) and Ar is aryl (e.g., phenyl having one or more non-hydrogen substituents such as C₁₋₆ alkyl, C₁₋₆ alkoxy, halogen, or methylenedioxy). The method includes reacting a masked mucohalic acid of Formula 1, 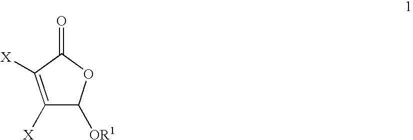
with an arylol of Formula 3,
Ar—OH 3
in the presence of a base and a solvent, but in the absence of Pd, to give the 4-aryloxy-2-buten-4-olide of Formula 2. In Formula 1 and Formula 3, X and Ar are as defined above for Formula 2, and OR¹ is a leaving group, which may include C₁₋₆ alkoxycarbonyloxy or C₁₋₆ alkanoyloxy. Useful bases include non-aqueous bases (e.g., CsF and KF), inorganic bases, such as Group 1 metal carbonates (e.g., Li₂CO₃, Na₂CO₃, NaHCO₃, K₂CO₃, and Cs₂CO₃) and phosphates (e.g., K₂HPO₄ and K₃PO₄), and other bases that are soluble in the solvent, such as NaOAc, NH₄OAc, alkyl₄NF, and the like. The reaction is typically carried out in an aprotic solvent, including 1,4-dioxane, THF, Et₂O, dichloromethane, trichloromethane, dichloroethane, nitromethane, ACN, NMP, DMF, DMSO, and the like.

Another aspect of the present invention provides a method of making compounds of Formula 4, 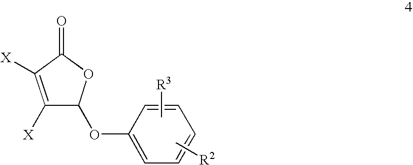
in which X is halogen (e.g., Cl or Br) and R² and R³ are independently H, C₁₋₆ alkyl, C₁₋₆ alkoxy, halogen, or together form a methylenedioxy. The method includes reacting the masked mucohalic acid of Formula 1 with a compound of Formula 5, 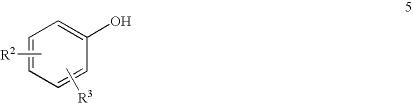
in the presence of a base and solvent, but in the absence of Pd, to give the 4-phenoxy-2-buten-4-olide of Formula 4. In Formula 5, substituents R² and R³ are as defined above for Formula 4, and in Formula 1, substituent X is halogen and R¹ is C₁₋₆ alkoxycarbonyl or C₁₋₆ alkanoyl. As in the method for preparing the compounds of Formula 2, useful bases include non-aqueous bases such as CsF and KF, inorganic bases, such as the Group 1 metal carbonates and phosphates, and other bases that are soluble in the solvent. Likewise, the reaction is typically carried out in an aprotic solvent, such as 1,4-dioxane, THF, Et₂O, dichloromethane, trichloromethane, dichloroethane, nitromethane, ACN, NMP, DMF, DMSO, alone or in combination.

A further aspect of the present invention provides a method of making compounds of Formula 6, 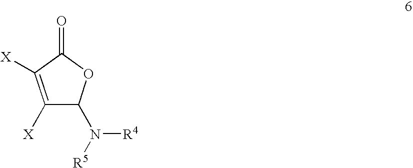
in which X is halogen (e.g., Cl or Br) and R⁴ and R⁵ are independently H, C₁₋₆ alkyl, C₁₋₆ alkenyl, aryl, or arylalkyl. The method includes reacting the masked mucohalic acid of Formula 1, 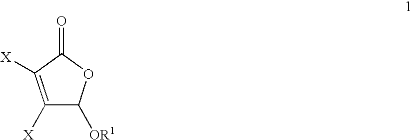
with a primary or secondary amine of Formula 7, 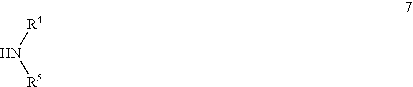
in the presence of a solvent (typically an aprotic solvent, including aromatic solvents, such as o-xylene, m-xylene, p-xylene, and toluene, or non-aromatic solvents, such as 1,4-dioxane, THF, Et₂O, EtOAc, dichloromethane, trichloromethane, dichloroethane, nitromethane, ACN, NMP, DMF, DMSO, and i-PrOH) and in the absence of Pd and other transition metals, to give the 4-amino-2-buten-4-olide of Formula 6. In Formula 7, substituents R⁴ and R⁵ are as defined above in Formula 6, and in Formula 1, X is halogen and R¹ is C-6 alkoxycarbonyl or C₁₋₆ alkanoyl.

An additional aspect of the present invention provides compounds of Formula 2, Formula 4, and Formula 6, as defined above, and compounds selected from:
4-methoxycarbonyloxy-2,3-dibromo-2-buten-4-olide;
4-t-butyloxycarbonyloxy-2,3-dichloro-2-buten-4-olide;
2,3-dichloro-4-m-tolyloxy-2-buten-4-olide;
2,3-dichloro-4-(4-methoxy-phenoxy)-2-buten-4-olide;
4-(benzo[1,3]dioxol-5-yloxy)-2,3-dichloro-2-buten-4-olide;
2,3-dichloro-4-phenoxy-2-buten-4-olide;
2,3-dichloro-4-(3-fluoro-phenoxy)-2-buten-4-olide;
2,3-dichloro-4-(3,5-dichloro-phenoxy)-2-buten-4-olide;
4-(3-bromo-phenoxy)-2,3-dichloro-2-buten-4-olide;
2,3-dibromo-4-m-tolyloxy-2-buten-4-olide;
4-benzylamino-2,3-dichloro-2-buten-4-olide;
4-allylamino-2,3-dichloro-2-buten-4-olide;
2,3-dichloro-4-dipropylamino-2-butene-4-olide;
4-benzylamino-2,3-dibromo-2-butene-4-olide;
4-methoxycarbonyloxy-3-anilino-2-bromo-2-buten-4-olide;
4-methoxycarbonyloxy-2-chloro-3-(pyridin-3-yloxy)-2-buten-4-olide;
3-chloro-4H,9aH-1,9-dioxa-4-aza-cyclopenta[b]naphthalen-2-one;
4-methoxycarbonyloxy-2-chloro-3-m-tolylsulfanyl-2-buten-4-olide; and
2,3-dichloro-4-(2-methyl-allyl)-2-buten-4-olide.

### DETAILED DESCRIPTION

Definitions and Abbreviations

Unless otherwise indicated, this disclosure uses definitions provided below. Some of the definitions and formulae may include a “-” (dash) to indicate a bond between atoms or a point of attachment to a named or unnamed atom or group of atoms. Other definitions and formulae may include an “=” to indicate a double bond.

“Substituted” groups are those in which one or more hydrogen atoms have been replaced with one or more non-hydrogen groups, provided that valence requirements are met and that a chemically stable compound results from the substitution.

“Alkyl” refers to straight chain and branched saturated hydrocarbon groups, generally having a specified number of carbon atoms (i.e., C₁₋₆ alkyl refers to an alkyl group having 1, 2, 3, 4, 5, or 6 carbon atoms). Examples of alkyl groups include, without limitation, methyl, ethyl, n-propyl, i-propyl, n-butyl, s-butyl, i-butyl, t-butyl, pent-1-yl, pent-2-yl, pent-3-yl, 3-methylbut-1-yl, 3-methylbut-2-yl, 2-methylbut-2-yl, 2,2,2-trimethyleth-1-yl, n-hexyl, and the like.

“Alkenyl” refers to straight chain and branched hydrocarbon groups having one or more unsaturated carbon-carbon bonds, and generally having a specified number of carbon atoms. Examples of alkenyl groups include, without limitation, ethenyl, 1-propen-1-yl, 1-propen-2-yl, 2-propen-1-yl, 1-buten-1-yl, 1-buten-2-yl, 3-buten-1-yl, 3-buten-2-yl, 2-buten-1-yl, 2-buten-2-yl, 2-methyl-1-propen-1-yl, 2-methyl-2-propen-1-yl, 1,3-butadien-1-yl, 1,3-butadien-2-yl, and the like.

“Alkynyl” refers to straight chain or branched hydrocarbon groups having one or more triple carbon-carbon bonds, and generally having a specified number of carbon atoms. Examples of alkynyl groups include, without limitation, ethynyl, 1-propyn-1-yl, 2-propyn-1-yl, 1-butyn-1-yl, 3-butyn-1-yl, 3-butyn-2-yl, 2-butyn-1-yl, and the like.

“Cycloalkyl” refers to saturated monocyclic and bicyclic hydrocarbon rings, generally having a specified number of carbon atoms that comprise the ring (i.e., C₃₋₇ cycloalkyl refers to a cycloalkyl group having 3, 4, 5, 6 or 7 carbon atoms as ring members and C₃₋₁₂ cycloalkyl refers to a cycloalkyl group having 3, 4, 5, 6, 7, 8, 9, 10, 11, or 12 carbon atoms as ring members). Cycloalkyl groups may be attached to a parent group or to a substrate at any ring atom, unless such attachment would violate valence requirements. Likewise, the cycloalkyl group may include one or more non-hydrogen substituents unless such substitution would violate valence requirements. Useful substituents include, without limitation, alkyl, alkoxy, alkoxycarbonyl, and alkanoyl, as defined above, and hydroxy, mercapto, nitro, halogen, and amino.

Examples of monocyclic cycloalkyl groups include, without limitation, cyclopropyl, cyclobutyl, cyclopentyl, cyclohexyl, and the like. Examples of bicyclic cycloalkyl groups include, without limitation, bicyclo[1.1.0]butyl, bicyclo[1.1.1]pentyl, bicyclo[2.1.0]pentyl, bicyclo[2.1.1]hexyl, bicyclo[3.1.0]hexyl, bicyclo[2.2.1]heptyl, bicyclo[3.2.0]heptyl, bicyclo[3.1.1]heptyl, bicyclo[4.1.0]heptyl, bicyclo[2.2.2]octyl, bicyclo[3.2.1]octyl, bicyclo[4.1.1]octyl, bicyclo[3.3.0]octyl, bicyclo[4.2.0]octyl, bicyclo[3.3.1]nonyl, bicyclo[4.2.1]nonyl, bicyclo[4.3.0]nonyl, bicyclo[3.3.2]decyl, bicyclo[4.2.2]decyl, bicyclo[4.3.1]decyl, bicyclo[4.4.0]decyl, bicyclo[3.3.3]undecyl, bicyclo[4.3.2]undecyl, bicyclo[4.3.3]dodecyl, and the like, which may be attached to a parent group or substrate at any of the ring atoms, unless such attachment would violate valence requirements.

“Alkanoyl” and “alkanoyloxy” refer, respectively, to alkyl-C(O)— and alkyl-C(O)—O—, where alkyl is defined above, and generally includes a specified number of carbon atoms, including the carbonyl carbon. Examples of alkanoyl groups include, without limitation, formyl, acetyl, propionyl, butyryl, pentanoyl, hexanoyl, and the like.

“Cycloalkanoyl” refers to cycloalkyl-C(O)—, where cycloalkyl is defined above, and generally includes a specified number of carbon atoms, excluding the carbonyl carbon. Examples of cycloalkanoyl groups include, without limitation, cyclopropanoyl, cyclobutanoyl, cyclopentanoyl, cyclohexanoyl, cycloheptanoyl, and the like.

“Alkoxy,” “alkoxycarbonyl,” and “alkoxycarbonyloxy” refer, respectively, to alkyl-O—, alkyl-O—C(O)—, and alkyl-O—C(O)—O—, where alkyl is defined above. Examples of alkoxy groups include, without limitation, methoxy, ethoxy, n-propoxy, i-propoxy, n-butoxy, s-butoxy, t-butoxy, n-pentoxy, s-pentoxy, and the like.

“Halo,” “halogen” and “halogeno” may be used interchangeably, and refer to fluoro, chloro, bromo, and iodo.

“Heterocycle” and “heterocyclyl” refer to saturated, partially unsaturated, or unsaturated monocyclic or bicyclic rings having from 5 to 7 or from 7 to 11 ring members, respectively. These groups have ring members made up of carbon atoms and from 1 to 4 heteroatoms that are independently nitrogen, oxygen or sulfur, and may include any bicyclic group in which any of the above-defined monocyclic heterocycles are fused to a benzene ring. The nitrogen and sulfur heteroatoms may optionally be oxidized. The heterocyclic ring may be attached to a parent group or to a substrate at any heteroatom or carbon atom unless such attachment would violate valence requirements. Likewise, any of the carbon or nitrogen ring members may include a non-hydrogen substituent unless such substitution would violate valence requirements. Useful substituents include, without limitation, alkyl, alkoxy, alkoxycarbonyl, alkanoyl, and cycloalkanoyl, as defined above, and hydroxy, mercapto, nitro, halogen, and amino.

Examples of heterocycles include, without limitation, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H, 6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

“Aryl” refers to aromatic groups, including heterocyclic groups as defined above, which are aromatic. Examples of aryl groups include, without limitation, phenyl, naphthyl, biphenyl, pyrenyl, anthracenyl, fluorenyl, pyrimidinyl, purinyl, imidazolyl, indolyl, quinolinyl isoquinolinyl, and the like, which may be unsubstituted or substituted with 1 to 4 substituents such as alkyl, alkoxy, alkoxycarbonyl, alkanoyl, and cycloalkanoyl, as defined above, and hydroxy, mercapto, oxy, nitro, halogen, and amino.

“Arylalkyl” refers to aryl-alkyl, where aryl and alkyl are defined above. Examples include, without limitation, benzyl, fluorenylmethyl, and the like.

“Leaving group” refers to any group that leaves a molecule during a fragmentation process, including substitution reactions, elimination reactions, and addition-elimination reactions. Leaving groups may be nucleofugal, in which the group leaves with a pair of electrons that formerly served as the bond between the leaving group and the molecule, or may be electrofugal, in which the group leaves without the pair of electrons. The ability of a nucleofugal leaving group to leave depends on its base strength, with the strongest bases being the poorest leaving groups. Common nucleofugal leaving groups include nitrogen (e.g., from diazonium salts), sulfonate esters (including tosylates, brosylates and mesylates), triflate esters, halide ions, carboxylate anions, phenolate ions, and alkoxides. Some stronger bases, such as NH₂⁻ and OH⁻ can be made better leaving groups by treatment with an acid. Common electrofugal leaving groups include the proton, CO₂, and metals.

“Absence,” as in the absence of Pd or the absence of Pd and other transition metals, means that the indicated material or materials are not present in a meaningful amount. For the purposes of this disclosure, the absence of Pd in a reaction mixture or the absence of Pd and other transition metals in a reaction mixture means that Pd or other transition metals are not purposely added to the reaction mixture. Therefore, Pd or each of the other transition metals may be present in trace amounts (e.g., about 50 ppm or less) but are not present in amounts that will substantially affect the outcome of the reaction.

Table 1 lists abbreviations, which are used throughout the specification.

TABLE 1
List of Abbreviations

| Abbreviation | Description |
|---|---|
| Ac | acetyl |
| ACN | acetonitrile |
| Aq | aqueous |
| Bn | benzyl |
| Bu | butyl |
| t-Bu | tertiary butyl |
| CO₂Me | methoxycarbonyloxy |
| CO₂t-Bu | tertiarybutoxycarbonyloxy |
| COMe | methylcarbonyl (acetyl) |
| d | day |
| DMF | dimethylformamide |
| DMSO | dimethylsulfoxide |
| Et | ethyl |
| ET₃N | triethylamine |
| EtOH | ethyl alcohol |
| Et₂O | ethyl ether |
| EtOAc | ethyl acetate |
| h | hour |
| Me | methyl |
| MeOH | methyl alcohol |
| min | minute |
| NaOAc | sodium acetate |
| NH₄OAc | ammonium acetate |
| NMP | N-methylpyrrolidone |
| NR | no reaction |
| 3-OCH₂O-4 | methylenedioxy |
| p-OMe | para-methoxy |
| PdCl₂(dppf)₂ | dichloro[1,1′-bis(diphenylphosphino)ferrocene] |
| | palladium (II) dichloromethane adduct |
| Pd₂(dba)₃ | tris(dibenzylidene-acetone)dipalladium(0) |
| Pd(PPh₃)₄ | tetrakis(triphenylphosphine)palladium(0) |
| Ph | phenyl |
| Ph₃P | triphenylphosphine |
| Pr | propyl |
| ppm | parts per million |
| i-Pr | isopropyl |
| i-PrOH | isopropyl alcohol |
| RT | room temperature (approximately 20° C. to 25° C.) |
| TFA | trifluoroacetic acid |
| THF | tetrahydrofuran |
| TLC | thin-layer chromatography |

Scheme I shows a palladium-free method of making 4-aryloxy-2-buten-4-olides of Formula 2. The method includes reacting a masked mucohalic acid of Formula 1 with an arylol of Formula 3 to give the 4-aryloxy-2-buten-4-olide of Formula 2. The reaction is carried out in the presence of a base and solvent, and in the absence of palladium. In Formula 1-3, X is halogen (Cl, Br, or I), OR¹ is a leaving group, and Ar is aryl. Useful X substituents include Cl and Br, and useful Ar substituents include phenyl, such as those having, in addition to a hydroxy substituent, one or more non-hydrogen substituents, including C₁₋₆ alkyl, C₁₋₆ alkoxy, halogen, or methylenedioxy (i.e., —OCH₂O—). Substituent OR¹, which is a better leaving group than X, is displaced during nucleophilic attack of the arylol on the masked mucohalic acid, and includes C₁₋₆ alkoxycarbonyloxy, C₁₋₆ alkanoyloxy, sulfonate ester (including tosylates, brosylates, mesylates, and triflates), OP(O)(O-aryl)₂, etc. Particularly useful OR¹ substituents include C₁₋₆ alkoxycarbonyloxy and C₁₋₆ alkanoyloxy.

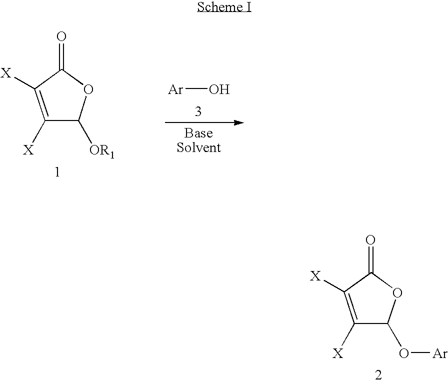

Although conversion of masked mucohalic acids to 4-aryloxy-2-buten-4-olides depends somewhat on the choice of base and solvent, a wide variety of bases and solvents can be used. Useful solvents include aprotic solvents, such as 1,4-dioxane, THF, Et₂O, dichloromethane, trichloromethane, dichloroethane, nitromethane, ACN, NMP, DMF, DMSO, and the like. Useful bases include non-aqueous bases (e.g., CsF and KF), inorganic bases, such as Group 1 metal carbonates (e.g., Li₂CO₃, Na₂CO₃, NaHCO₃, K₂CO₃, and Cs₂CO₃) and phosphates (e.g., K₂HPO₄ and K₃PO₄), and other bases that are soluble in the solvent, such as NaOAc, NH₄OAc, alkyl₄NF, and the like. Generally, catalytic amounts of base (e.g., from about 15 mol % to about 20 mol %) are sufficient to effect the transformation shown in Scheme I. For less efficient leaving groups (e.g., when OR¹ is C₁₋₆ alkanoyloxy) one or more equivalents of base may be used.

The conversion of masked mucohalic acids to 4-aryloxy-2-buten-4-olides can be undertaken using substantially stoichiometric amounts of reactants, though it may be advantageous to carryout the reaction with an excess of the arylol (e.g., from about 1.1 equivalents to about 1.5 equivalents). More generally, and unless stated otherwise, the chemical transformations described throughout the specification can be carried out using substantially stoichiometric amounts of reactants or using an excess of one or more of the reactants. In addition, and unless stated otherwise, any reference in the disclosure to a stoichiometric range, a temperature range, a pH range, etc., includes the indicated endpoints.

As shown in the examples below, the reaction between various masked mucohalic acids and arylols at room temperature (RT) results in good yields of 4-aryloxy-2-buten-4-olides. Moreover, all of the conversions occur within a reasonable period of time (i.e., reaction times under about 24 hours). To modify reaction time and yield, however, the reaction temperature may be varied from about −10° C. to about 60° C.

Scheme II shows a palladium-free method of making 4-amino-2-buten-4-olides of Formula 6. The method includes reacting a masked mucohalic acid of Formula 1 with a primary or secondary amine of Formula 7 to give the 4-amino-2-buten-4-olide of Formula 6. The reaction is carried out in a solvent (typically an aprotic solvent, including aromatic solvents, such as o-xylene, m-xylene, p-xylene, and toluene, or non-aromatic solvents, such as 1,4-dioxane, THF, Et₂O, EtOAc, dichloromethane, trichloromethane, dichloroethane, nitromethane, ACN, NMP, DMF, DMSO, and i-PrOH) and in the absence of a palladium and other transition metals. In Formula 1, 6, and 7, substituents X and OR¹ are as defined above for Scheme I, and substituents R⁴ and R⁵ are independently H, C₁₋₆ alkyl, C₁₋₆ alkenyl, aryl, and arylalkyl.

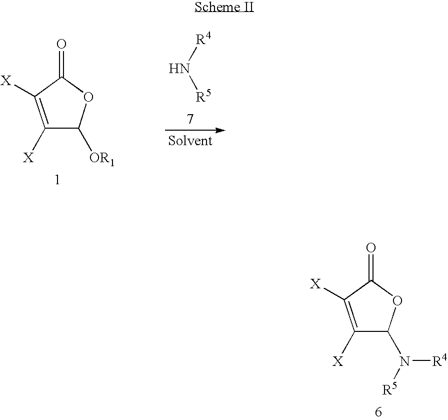

Like the transformation shown in Scheme I, the conversion of masked mucohalic acids to 4-amino-2-buten-4-olides shown in Scheme II can be undertaken using substantially stoichiometric amounts of reactants, though it may be advantageous to carryout the reaction with an excess of the amine (e.g., from about 1.1 equivalents to about 2.0 equivalents). Similarly, as shown in the examples, the reaction between various masked mucohalic acids and amines at temperatures between about 0° C. and RT generally result in good yields of 4-amino-2-buten-4-olides within a period of time (i.e., reaction times under about 17 hours). To modify reaction time and yield, however, the reaction temperature may be varied from about −25° C. to about 50° C.

The reaction of the masked mucohalic acid of Formula 1 with an arylol of Formula 3 or Formula 5 or with a primary or secondary amine of Formula 7, results in highly regioselective nucleophilic displacement of the masked mucohalic acid without the use of a palladium catalyst. Indeed, the yields and reaction times of the palladium-free conversions of the masked mucohalic acids to 4-aryloxy- or 4-amino-2-buten-4-olides at RT are comparable to the yields and reactions times of Pd-catalyzed allylic conversions (compare Example 5-9 with Example 10-13). This result is surprising since numerous research reports indicate that palladium catalysis is needed to differentiate the allylic and vinylic functionalities of masked mucohalic acids.

The masked mucohalic acids of Formula 1 may be prepared in a number of ways. For example, mucochloric acid or mucobromic acid can be reacted with a C₁₋₆ alkoxycarbonylchloride, such as methyl chloroformate, in the presence of a non-nucleophilic base, such as diisopropylethylamine, and an aprotic solvent, such as dichloromethane, to yield a 2,3-dihalo-4-C₁₋₆ alkoxycarbonyloxy-2-buten-4-olide. Similarly, mucochloric acid or mucobromic acid can be reacted with an acid chloride or acid anhydride, such as acetyl chloride or acetic anhydride, in the presence of a non-nucleophilic base and an aprotic solvent, to yield a 2,3-dihalo-4-alkanoyloxy-2-buten-4-olide.

Some of the compounds disclosed in this specification may contain one or more asymmetric carbon atoms and therefore may exist as optically active stereoisomers (i.e., pairs of enantiomers). Some of the compounds may also contain an alkenyl or cyclic group, so that cis/trans (or Z/E) stereoisomers (i.e., pairs of diastereoisomers) are possible. Still other compounds may exist as one or more pairs of diastereoisomers in which each diastereoisomer exists as one or more pairs of enantiomers. Finally, some of the compounds may contain a keto or oxime group, so that tautomerism may occur. In such cases, the scope of the present invention includes individual stereoisomers of the disclosed compound, as well as its tautomeric forms (if appropriate).

Individual enantiomers may be prepared or isolated by known techniques, such as conversion of an appropriate optically-pure precursor, resolution of the racemate (or the racemate of a salt or derivative) using, for example, chiral HPLC, or fractional crystallization of diastereoisomeric salts formed by reaction of the racemate with a suitable optically active acid or base (e.g., tartaric acid). Diastereoisomers may be separated by known techniques, such as fractional crystallization and chromatography.

The disclosed compounds also include all isotopic variations, in which at least one atom is replaced by an atom having the same atomic number, but an atomic mass different from the atomic mass usually found in nature. Examples of isotopes suitable for inclusion in the disclosed compounds include, without limitation, isotopes of hydrogen, such as ²H and ³H; isotopes of carbon, such as ¹³C and ¹⁴C; isotopes of nitrogen, such as ¹⁵N; isotopes of oxygen, such as ¹⁷O and ¹⁸O; isotopes of phosphorus, such as ³¹P and ³²P; isotopes of sulfur, such as ³⁵S; isotopes of fluorine, such as ¹⁸F; and isotopes of chlorine, such as ³⁶Cl.

### EXAMPLES

The following examples are intended to be illustrative and non-limiting, and represent specific embodiments of the present invention.

General Methods

All reactions were carried out under nitrogen atmosphere unless otherwise noted. All solvents and reagents used were from commercial sources and no further purification was performed. Reactions were monitored by mass spectrometry (MS) on a Micromass Platform LC and by thin-layer chromatography on 0.25 mm E. Merck silica gel 60 plates (F₂₅₄) using UV light and aqueous potassium permanganate-sodium bicarbonate as visualizing agents. E. Merck silica gel 60 (0.040-0.063 mm and 0.063-0.200 mm particle sizes) was used for column chromatography. Proton nuclear magnetic resonance (¹H NMR) spectra were recorded at 400 MHz on a Varian UNITY INOVA AS400. Chemical shifts are reported as delta (6) units in parts per million (ppm) relative to the singlet at 7.26 ppm for deuteriochloroform or the singlet at 4.81 ppm for methanol-d₆. Coupling constants (J) are reported in Hertz (Hz). Carbon-13 nuclear magnetic resonance (¹³C NMR) spectra were recorded at 100 MHz on a Varian UNITY Plus INOVA 400. Chemical shifts are reported as delta (6) units in parts per million (ppm) relative to the center line of the triplet at 77.3 ppm for deuteriochloroform or the center line of the septet at 47.8 ppm for methanol-d₆. Elemental analyses were performed by Quantitative Technologies Inc.

### Example 1

### Preparation of 4-methoxycarbonyloxy-2,3-dichloro-2-buten-4-olide

Methyl chloroformate (9.92 g, 105 mmol) was added to a cold (−10° C. to −5° C.) solution of mucochloric acid (16.9 g, 100 mmol) in dry dichloromethane (200 mL). Diisopropylethylamine (14.2 g, 110 mmol) was then added over a 5 min period and the resulting mixture was stirred at −10° C. to −5° C. for 2.5 h. The reaction mixture was quenched with water (200 mL) and diluted with dichloromethane (600 mL). The phases were separated and the red organic phase was washed with water (400 mL). The organic phase was concentrated under reduced pressure. The residue was purified by silica gel column chromatography (ethyl acetate/heptane) followed by recrystallization (ethyl acetate/heptane) to give 4-methoxycarbonyloxy-2,3-dichloro-2-buten-4-olide as an off-white solid: yield 16.2 g (71%). ¹H NMR (CDCl₃): δ 6.76 (s, 1H), 3.93 (s, 3H). ¹³C NMR (CDCl₃): δ 162.4, 153.6, 146.4, 125.3, 94.4, 56.6. Anal. Calc'd for C₆H₄Cl₂O₅: C, 31.75; H, 1.78; Cl, 31.24. Found: C, 31.75; H, 1.57; Cl, 31.48.

### Example 2

### Preparation of 4-methoxycarbonyloxy-2,3-dibromo-2-buten-4-olide

Methyl chloroformate (4.96 g, 52.5 mmol) was added to a cold (−10° C. to −5° C.) solution of mucobromic acid (12.9 g, 50.0 mmol) in dry dichloromethane (200 mL). Diisopropylethylamine (7.11 g, 55.5 mmol) was then added over a 10 min period and the resulting mixture was stirred at −10° C. to −5° C. for 1.5 h. The reaction mixture was quenched with water (100 mL) and diluted with dichloromethane (200 mL). The phases were separated and the red organic phase was concentrated under reduced pressure. The residue was purified by silica gel column chromatography (ethyl acetate/heptane) followed by recrystallization (ethyl acetate/heptane) to give 4-methoxycarbonyloxy-2,3-dibromo-2-buten-4-olide as a beige solid: yield 7.17 g (45%). ¹H NMR (CDCl₃): δ 6.76 (s, 1H), 3.93 (s, 3H). ¹³C NMR (CDCl₃): δ 163.3, 153.6, 141.6, 119.6, 96.6, 56.5. Anal. Calc'd for C₆H₄Br₂O₅: C, 22.81; H, 1.28; Br, 50.59. Found: C, 22.75; H, 1.23; Br, 50.88.

### Example 3

### Preparation of 4-t-butyloxycarbonyloxy-2,3-dichloro-2-buten-4-olide

Mucochloric acid (1.69 g, 10.0 mmol), di-t-butyl dicarbonate (2.50 g, 11.5 mmol) and N-methylmorpholine (1.21 g, 12.0 mmol) were combined in dry dichloromethane (50 mL) and stirred at RT for 18 h. The reaction mixture was quenched with water (50 mL) and partitioned between water (20 mL) and dichloromethane (50 mL). The phases were separated and the dark organic phase was concentrated under reduced pressure. The residue was purified by silica gel column chromatography (ethyl acetate/heptane) followed by pulping in heptane to give 4-t-butyloxycarbonyloxy-2,3-dichloro-2-buten-4-olide as a white solid: yield 0.93 g (35%). ¹H NMR (CDCl₃): δ 6.73 (s, 1H), 1.54 (s, 9H). ¹³C NMR (CDCl₃): δ 162.7, 150.8, 146.7, 93.9, 86.1, 27.8. Anal. Calc'd for C₉H₁₀Cl₂O₅: C, 40.17; H, 3.75; Cl, 26.35. Found: C, 40.33; H, 3.54; Cl, 26.62.

### Example 4

### Preparation of 4-acetoxy-2,3-dichloro-2-buten-4-olide

Mucochloric acid (16.9 g, 100 mmol), acetic anhydride (12.3 g, 120 mmol) and Amberlyst-15 (0.7 g) were combined in dry dichloromethane (300 mL) and stirred at RT for 3 d. The reaction mixture was diluted with dichloromethane (500 mL) and washed with saturated aqueous sodium bicarbonate (3×200 mL) and brine (100 mL). The organic phase was dried over magnesium sulfate and concentrated under reduced pressure to give 4-acetoxy-2,3-dichloro-2-buten-4-olide as a colorless oil: yield 20.4 g (97%). ¹H NMR (CDCl₃): δ 6.90 (s, 1H), 2.22 (s, 3H). ¹³C NMR (CDCl₃): δ 168.6, 162.8, 147.2, 124.7, 91.4, 20.7. Anal. Calc'd for C₆H₄Cl₂O₄: C, 34.15; H, 1.91; Cl, 33.60. Found: C, 34.24; H, 1.88; Cl, 33.41.

### Example 5-13

### Preparation of 2,3-dichloro-4-m-tolyloxy-2-buten-4-olide (Formula 8)

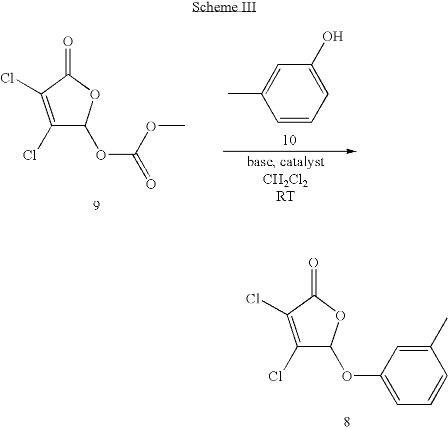

Table 2 lists catalyst, base, and reaction times for various etherifications of 4-methoxycarbonyloxy-2,3-dichloro-2-buten-4-olide (Formula 9). The masked mucohalic acid of Formula 9 (2.0 mmol) was combined with m-cresol (Formula 10, 1.1 equiv.), base (0.15-0.50 equiv.), Pd catalyst (0 or 3 mol %), and dichloromethane (20 mL) and was stirred at RT for 4 h to 65 h, depending on the reaction conditions. The reaction was quenched with saturated aqueous ammonium chloride (20 mL) and partitioned between water (20 μL) and dichloromethane (80 mL). The organic extract was concentrated under reduced pressure. The residue was purified by silica gel column chromatography (ethyl acetate/heptane) to give 2,3-dichloro-4-m-tolyloxy-2-buten-4-olide in yields shown in Table 2. ¹H NMR (CDCl₃): δ 7.21-7.25 (m, 1H), 6.92-6.98 (m, 3H), 6.21 (s, 1H), 2.35 (s, 3H). ¹³C NMR (CDCl₃): δ 163.0, 156.0, 147.2, 140.5, 129.9, 125.7, 124.9, 118.4, 114.7, 99.5, 21.6. Anal. Calc'd for C₁₁h₈Cl₂O₃: C, 50.99; H, 3.11; Cl, 27.37. Found: C, 51.22; H, 3.13; Cl, 27.05.

TABLE 2
Preparation of 2,3-dichloro-4-m-tolyloxy-2-buten-4-olide via
etherification of 4-methoxycarbonyloxy-2,
3-dichloro-2-buten-4-olide with m-cresol

| Exam- | | | | |
|---|---|---|---|---|
| ple | Catalyst | Base | Time^{a}, h | Yield^{b} |
| 5 | Pd₂dba₃ (3 mol %) | Cs₂CO₃ (15 mol %) | 4 | 83 |
| 6 | Pd₂dba₃ (3 mol %) | CsF (20 mol %) | 7 | 87 |
| 7 | Pd₂dba₃ (3 mol %) | KF (20 mol %) | 48 | 50 |
| 8 | Pd(PPh₃)₄ (3 mol %) | Cs₂CO₃ (15 mol %) | 2.5 | 69 |
| 9 | Pd₂dba₃ (3 mol %) | None | 65 | NR^{c} |
| 10 | None | Cs₂CO₃ (15 mol %) | 0.5 | 75 |
| 11 | None | Cs₂CO₃ (15 mol %) | 7.5 | 87 |
| 12 | None | Na₂CO₃ (50 mol %) | 48 | NR |
| 13 | None | CsF (20 mol %) | 7 | 89 |

^{a}Reaction times were not optimized
^{b}Isolated Yields
^{c}No reaction observed

### Example 14-22

### Preparation of 4-aryloxy-2-buten-4-olides (Formula 4) via etherification of masked mucohalic acids (Formula 1) with substituted phenols (Formula 5)

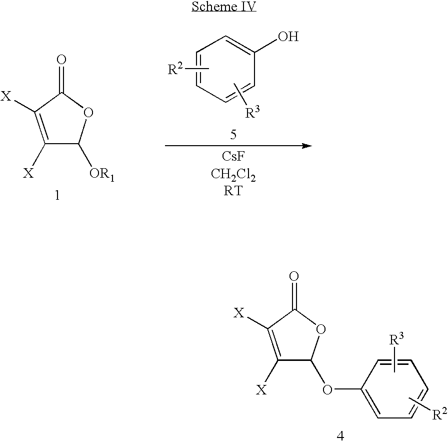

Table 3 lists reaction times and yields for preparing various 4-aryloxy-2-buten-4-olides (Formula 4) through etherification of masked mucohalic acids (Formula 1). A masked mucohalic acid of Formula 1 (2.0 mmol) was combined with a substituted phenol (Formula 5, 1.1 equiv.), CsF (0.2 or 1.1 equiv.) and dichloromethane (20 mL) and was stirred at RT for 7 h to 24 h. The reaction was quenched with saturated aqueous ammonium chloride (20 mL) and partitioned between water (20 mL) and dichloromethane (80 mL). The organic extract was concentrated under reduced pressure. The residue was purified by silica gel column chromatography (ethyl acetate/heptane) to give various 4-aryloxy-2-buten-4-olides in yields shown in Table 3.

Example 14. 2,3-Dichloro-4-(4-methoxy-phenoxy)-2-buten-4-olide. ¹H NMR (CDCl₃): δ 7.07-7.11 (m, 2H), 6.85-6.89 (m, 2H), 6.14 (s, 1H), 3.79 (s, 3H). ¹³C NMR (CDCl₃): δ 163.0, 157.0, 149.5, 147.2, 125.0, 119.8, 115.1, 100.4, 55.9. Anal. Calc'd for C₁₁H₈Cl₂O₄: C, 48.03; H, 2.93; Cl, 25.78. Found: C, 47.93; H, 2.78; Cl, 25.55.

Example 15. 4-(Benzo[1,3]dioxol-5-yloxy)-2,3-dichloro-2-buten-4-olide. ¹H NMR (CDCl₃): δ 6.61-6.75 (m, 3H), 6.11 (s, 1H), 5.98 (s, 2H). ³C NMR (CDCl₃): δ 162.9, 150.7, 148.6, 147.0, 145.1, 125.1, 110.8, 108.4, 102.0, 101.5, 100.4. Anal. Calc'd for C₁₁H₆Cl₂O₅: C, 45.71; H, 2.09; Cl, 24.53. Found: C, 45.78; H, 1.90; Cl, 24.76.

TABLE 3
Preparation of 4-aryloxy-2-buten-4-olides (Formula 4)
via etherification of masked mucohalic acids (Formula 1)
with substituted phenols (Formula 5)

| | | | | | CsF | Time^{a}, | |
|---|---|---|---|---|---|---|---|
| Example | X | R¹ | R² | R³ | Equiv. | h | Yield^{b} |
| 14 | Cl | CO₂Me | p-OMe | H | 0.2 | 7 | 91 |
| 15 | Cl | CO₂Me | 3-OCH₂O-4 | | 0.2 | 7 | 86 |
| 16 | Cl | CO₂Me | H | H | 0.2 | 7 | 88 |
| 17 | Cl | CO₂Me | m-F | H | 0.2 | 23 | 72 |
| 18 | Cl | CO₂Me | 3-Cl | 5-Cl | 0.2 | 23 | 70 |
| 19 | Cl | CO₂Me | m-Br | H | 0.2 | 24 | 62 |
| 20 | Br | CO₂Me | m-Me | H | 0.2 | 6 | 91 |
| 21 | Cl | CO₂t-Bu | m-Me | H | 0.2 | 6 | 81 |
| 22 | Cl | COMe | m-Me | H | 1.1 | 22 | 75 |

^{a}Reaction times were not optimized
^{b}Isolated Yields

Example 16. 2,3-Dichloro-4-phenoxy-2-buten-4-olide. ¹H NMR (CDCl₃): δ 7.35-7.40 (m, 2H), 7.14-7.21 (m, 3H), 6.25 (s, 1H), 3.79. ¹³C NMR (CDCl₃): δ 163.0, 155.9, 147.0, 130.2, 125.0, 117.8, 99.4. Anal. Calc'd for C₁₀H₆Cl₂O₃: C, 49.01; H, 2.47; Cl, 28.93. Found: C, 49.02; H, 2.26; Cl, 28.81.

Example 17. 2,3-Dichloro-4-(3-fluoro-phenoxy)-2-buten-4-olide. ¹H NMR (CDCl₃): δ 7.30-7.36 (m, 1H), 6.87-6.97 (m, 3H), 6.23 (s, 1H). ¹³C NMR (CDCl₃): δ 164.7, 162.8, 162.3, 156.9, 156.8, 146.9, 131.2, 131.1, 125.2, 113.2, 113.1, 112.1, 111.9, 106.0, 105.8, 98.9. Anal. Calc'd for C₁₀H₅Cl₂F₁O₃: C, 45.66; H, 1.92; Cl, 26.95; F, 7.22. Found: C, 45.63; H, 1.65; Cl, 27.10; F, 7.53.

Example 18. 2,3-Dichloro-4-(3,5-dichloro-phenoxy)-2-buten-4-olide. ¹H NMR (CDCl₃): δ 7.13-7.25 (m, 1H), 7.07-7.08 (m, 2H), 6.21 (s, 1H). ¹³C NMR (CDCl₃): δ 165.5, 156.6, 146.7, 136.2, 125.3, 120.6, 116.7, 98.6. Anal. Calc'd. for C₁₀H₄Cl₄O₃: C, 38.26; H, 1.28; Cl, 45.17. Found: C, 38.64; H, 1.21; Cl, 45.35.

Example 19. 4-(3-Bromo-phenoxy)-2,3-dichloro-2-buten-4-olide. ¹H NMR (CDCl₃): δ 7.30-7.33 (m, 2H), 7.21-7.25 (m, 1H), 7.07-7.11 (m, 1H), 6.21 (s, 1H). C NMR (CDCl₃): 6162.8, 156.5, 146.9, 131.4, 128.2, 125.2, 123.3, 121.2, 116.4, 99.0. Anal. Calc'd for C₁₀H₅Br₁Cl₂O₃: C, 37.08; H, 1.56; Br, 24.67. Found: C, 37.16; H, 1.29; Br, 25.04.

Example 20. 2,3-Dibromo-4-m-tolyloxy-2-buten-4-olide. ¹H NMR (CDCl₃): δ 7.22-7.25 (m, 1H), 6.95-6.99 (m, 3H), 6.22 (s, 1H), 2.36 (s, 3H). ¹³C NMR (CDCl₃): δ 164.0, 156.1, 142.9, 140.5, 129.9, 125.7, 119.3, 118.4, 114.6, 101.7, 21.7. Anal. Calc'd for C₁₁H₈Br₂O₃: C, 37.97; H, 2.32; Br, 45.92. Found: C, 38.34; H, 2.15; Br, 46.06.

### Example 23-28

### Preparation of 4-amino-2-buten-4-olides (Formula 6) via Amination of Masked Mucohalic Acids (Formula 1) with Primary and Secondary Amines (Formula 7)

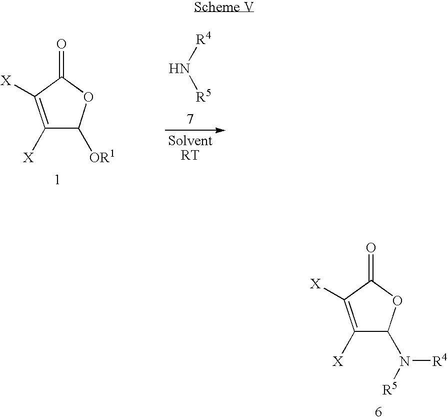

Table 4 lists reaction times and yields for preparing various 4-amino-2-buten-4-olides (Formula 6) through amination of masked mucohalic acids (Formula 1). A masked mucohalic acid of Formula 1 (2.0 mmol) was combined with a primary or secondary amine (Formula 7, 1.0-2.0 equiv.) and toluene (20 mL) and was stirred at 0° C. to 25° C. for 2 h to 8 h. The reaction was partitioned between brine (10 mL) and toluene (20 mL). The organic phase was concentrated under reduced pressure, and the residue was purified by silica gel column chromatography (ethyl acetate/heptane) to give various 4-amino-2-buten-4-olides in yields shown in Table 4.

TABLE 4
Preparation of 4-amino-2-buten-4-olides (Formula 6)
via amination of masked mucohalic acids (Formula 1)
with 1° or 2° amines (Formula 7)

| Ex- | | | | | | | | |
|---|---|---|---|---|---|---|---|---|
| am- | | | | | Amine | Temp. | Time^{a}, | |
| ple | X | R¹ | R⁴ | R⁵ | Equiv. | ° C. | h | Yield^{b} |
| 23 | Cl | CO₂Me | Bn | H | 1.1 | 20-25 | 5 | 79 |
| 24 | Cl | CO₂Me | Allyl | H | 1.1 | 20-25 | 2 | 74 |
| 25 | Cl | CO₂Me | n-Pr | n-Pr | 1.1 | 20-25 | 17 | 61 |
| 26 | Cl | COMe | n-Pr | n-Pr | 1.1 | 20-25 | 25 | 54 |
| 27 | Br | CO₂Me | Bn | H | 1.1 | 0→25 | 5 | 65 |
| 28 | Cl | COMe | Bn | H | 2.0 | 0→25 | 8 | 46 |

^{a}Reaction times were not optimized
^{b}Isolated Yields

Example 23. 4-Benzylamino-2,3-dichloro-2-buten-4-olide. ¹H NMR (CDCl₃): δ 7.28-7.36 (m, 5H), 5.11 (d, J=10.7, 1H), 4.98 (d, J=14.9, 1H), 4.29 (d, J=14.9, 1H), 3.45 (variable) (d, J=10.7, 1H). ¹³C NMR (CDCl₃): δ 163.3, 144.4, 135.9, 129.2, 128.7, 128.3, 126.2, 81.6, 43.9. Anal. Calc'd for C₁₁H₉Cl₂N₁O₂: C, 51.19; H, 3.51; Cl, 27.47; N, 5.43. Found: C, 51.47; H, 3.35; Cl, 27.25; N, 5.23.

Example 24. 4-Allylamino-2,3-dichloro-2-buten-4-olide. ¹HNMR (CDCl₃): δ 5.72-5.81 (m, 1H), 5.20-5.28 (m, 3H), 4.26-4.32 (m, 1H), 3.91 (variable) (d, J=9.0, 1H), 3.79-3.85 (m, 1H). ¹³C NMR (CDCl₃): δ 163.0, 144.1, 131.9, 126.6, 81.9, 42.8. Anal. Calc'd for C₇H₇Cl₂N₁O₂: C, 40.41; H, 3.39; Cl, 34.08; N, 6.73. Found: C, 40.50; H, 3.21; Cl, 33.94; N, 6.80.

Example 25. 2,3-Dichloro-4-dipropylamino-2-butene-4-olide. ¹H NMR (CDCl₃): δ 9.53 (s, 1H), 5.053.38-3.42 (m, 1H), 3.18-3.21 (m, 1H), 1.56-1.70 (m, 2H), 0.94 (t, J=7.3, 1H), 0.88 (t, J=7.3, 1H). ¹³C NMR (CDCl₃): δ 181.1, 161.8, 145.5, 134.0, 50.6, 46.9, 21.8, 20.5, 11.6, 11.4. Anal. Calc'd for C₁₀H₁₅Cl₂N₁O₂: C, 47.64; H, 6.00; Cl, 28.12; N, 5.56. Found: C, 47.30; H, 5.79; Cl, 28.31; N, 5.63.

Example 27. 4-Benzylamino-2,3-dibromo-2-butene-4-olide. ¹HNMR (CDCl₃): δ 7.28-7.36 (m, 5H), 5.12 (d, J=10.7, 1H), 4.99 (d, J=14.9, 1H), 4.32 (d, J=14.6, 1H), 3.46 (variable) (d, J=11.0, 1H). ¹³C NMR (CDCl₃): δ 163.6, 139.6, 136.1, 129.3, 128.9, 128.4, 122.4, 84.0, 44.5. Anal. Calc'd for C₁₁H₉Br₂N₁O₂: C, 38.07; H, 2.61; Br, 46.05; N, 4.04. Found: C, 38.16; H, 2.53; Br, 46.28; N, 4.15.

### Example 29

### Preparation of 4-methoxycarbonyloxy-3-anilino-2-bromo-2-buten-4-olide

A masked mucohalic acid, 4-methoxycarbonyloxy-2,3-dibromo-2-buten-4-olide (1.0 mmol) was combined with aniline (2.0 equiv.) in NMP (5 mL) and stirred at RT for 24 h. The reaction mixture was diluted with water (50 μL) and the resulting slurry was filtered. The solid was washed with water (10 mL) and heptane (5 mL) and dried to give, without further purification, 4-methoxycarbonyloxy-3-anilino-2-bromo-2-buten-4-olide. ¹H NMR (CDCl₃): δ 7.37-7.42 (m, 2H), 7.27-7.31 (m, 1H), 7.15-7.18 (m, 2H), 6.92 (s, 1H), 6.74 (br s, 1H), 3.72 (s, 3H). ¹³C NMR (CDCl₃): δ 166.2, 156.7, 153.6, 136.2, 129.9, 127.5, 124.6, 92.5, 79.5, 56.1. Anal. Calc'd for C₁₂H₁₀Br₁N₁O₅: C, 43.93; H, 3.07; Br, 24.35; N, 4.27. Found: C, 43.38; H, 2.85; Br, 24.75; N, 4.30.

### Example 30

### Preparation of 4-methoxycarbonyloxy-2-chloro-3-(pyridin-3-yloxy)-2-buten-4-olide

A masked mucohalic acid, 4-methoxycarbonyloxy-2,3-dichloro-2-buten-4-olide (2.0 mmol) was combined with 3-hydroxypyridine (1.1 equiv.) and CsF (0.2 equiv.) in dichloromethane (20 mL) and stirred at RT for 22 h. The reaction mixture was concentrated under reduced pressure, and the residue was purified by silica gel column chromatography (ethyl acetate/heptane) to give 4-methoxycarbonyloxy-2-chloro-3-(pyridin-3-yloxy)-2-buten-4-olide. ¹H NMR (CDCl₃): δ 8.57-8.60 (m, 2H), 7.52-7.55 (m, 1H), 7.39-7.42 (m, 1H), 6.83 (s, 1H), 3.90 (s, 3H). ¹³C NMR (CDCl₃): δ 164.4, 161.7, 153.6, 149.6, 148.5, 142.3, 127.7, 124.5, 101.5, 56.5. Anal. Calc'd for C₁₁H₈C₁₁N₁O₆: C, 46.25; H, 2.82; Cl, 12.41; N, 4.90. Found: C, 46.20; H, 2.76; Cl, 12.67; N, 4.80.

### Example 31

### Preparation of 3-chloro-4H,9aH-1,9-dioxa-4-aza-cyclopenta[b]naphthalen-2-one

A masked mucohalic acid, 4-methoxycarbonyloxy-2,3-dichloro-2-buten-4-olide (1.0 mmol) was combined with 2-aminophenol (1.1 equiv.) and CsF (1.1 equiv.) in dichloromethane (10 mL) and tetrahydrofuran (5 mL) and stirred at RT for 41 h. The reaction mixture was partitioned between saturated aqueous sodium chloride (15 mL) and dichloromethane (25 mL). The organic phase was concentrated under reduced pressure. The residue was purified by silica gel column chromatography (ethyl acetate/heptane) to give 3-chloro-4H,9aH-1,9-dioxa-4-aza-cyclopenta[b]naphthalen-2-one. ¹H NMR (CD₃OD): δ 6.97-7.05 (m, 4H), 5.95 (s, 1H). ¹³C NMR (CD₃OD): δ 167.5, 150.4, 141.0, 127.3, 124.3, 124.2, 118.0, 116.9, 92.2, 47.8. Anal. Calc'd for C₁₀H₆C₁₁N₁O₃: C, 53.71; H, 2.70; Cl, 15.85; N, 6.26. Found: C, 53.50; H, 3.00; Cl, 16.09; N, 6.28.

### Example 32

### Preparation of 4-methoxycarbonyloxy-2-chloro-3-m-tolylsulfanyl-2-buten-4-olide

A masked mucohalic acid, 4-methoxycarbonyloxy-2,3-dichloro-2-buten-4-olide (2.0 mmol) was combined with m-thiocresol (1.1 equiv.) and CsF (0.7 equiv.) in dichloromethane (20 mL) and stirred at RT for 3 d. The reaction was quenched with saturated aqueous ammonium chloride (20 mL) and partitioned between water (20 mL) and dichloromethane (80 mL). The organic extract was concentrated under reduced pressure. The residue was purified by silica gel column chromatography (ethyl acetate/heptane) to give 4-methoxycarbonyloxy-2-chloro-3-m-tolylsulfanyl-2-buten-4-olide. ¹H NMR (CDCl₃): δ 7.38-7.41 (m, 2H), 7.29-7.31 (m, 2H), 6.46 (s, 1H), 3.69 (s, 3H), 2.37 (s, 3H). ¹³C NMR (CDCl₃): δ 163.7, 154.9, 153.2, 140.4, 136.6, 133.0, 132.0, 129.9, 124.1, 117.4, 94.0, 55.9, 21.4. Anal. Calc'd for C₁₃H₁₁Cl₁O₅S₁: C, 49.61; H, 3.52; Cl, 11.26; S, 10.19. Found: C, 49.34; H, 3.28; Cl, 11.12; S, 10.21.

### Example 33

### Preparation of 2,3-dichloro-4-(2-methyl-allyl)-2-buten-4-olide

Indium powder (1.2 equiv., −100 mesh, 99.99%) was combined with 3-bromo-2-methylpropene (1.2 equiv.) in THF (10 mL) and NMP (1 mL) and stirred at RT for 1 h. A masked mucohalic acid, 4-methoxycarbonyloxy-2,3-dichloro-2-buten-4-olide (5.0 mmol) was added and the reaction mixture was stirred at RT for 24 h. The reaction mixture was concentrated under reduced pressure, and the residue was purified by silica gel column chromatography (ethyl acetate/heptane) to give 3-dichloro-4-(2-methyl-allyl)-2-buten-4-olide. ¹H NMR (CDCl₃): δ 5.08 (dd, J=8.1, J=3.7, 1H), 4.95 (t, J=1.5, 1H), 4.87 (s, 1H), 2.75 (dd, J=14.9, J=3.4, 1H), 2.32-2.40 (m, 1H), 1.80 (s, 3H). ¹³C NMR (CDCl₃): δ 165.3, 152.5, 138.4, 121.4, 115.8, 81.3, 39.8, 23.2. Anal. Calc'd for C₈H₈Cl₂O₂: C, 46.41; H, 3.89. Found: C, 46.70; H, 3.86.

It is to be understood that the above description is intended to be illustrative and not restrictive. Many embodiments will be apparent to those of skill in the art upon reading the above description. The scope of the invention should, therefore, be determined not with reference to the above description, but should instead be determined with reference to the appended claims, along with the full scope of equivalents to which such claims are entitled. The disclosures of all articles and references, including patent applications, granted patents, and publications, are incorporated herein by reference in their entirety and for all purposes.

1. A method of making a compound of Formula 2, 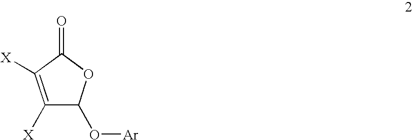
wherein X is halogen and Ar is aryl, the method comprising:
reacting a compound of Formula 1, 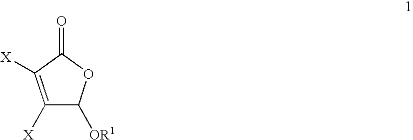
with a compound of Formula 3,
Ar—OH 3
in the presence of a base and in the absence of Pd, wherein X and Ar are as defined above for Formula 2, and OR¹ is a leaving group.

2. The method of claim 1, further comprising providing the compound of Formula 1 in which the leaving group is C₁₋₆ alkoxycarbonyloxy or C₁₋₆ alkanoyloxy.

3. The method of claim 1, further comprising providing the compound of Formula 1 in which X is chlorine or bromine.

4. The method of claim 1, further comprising providing the compound of Formula 3 in which Ar is a phenyl group, the phenyl group having, in addition to a hydroxy substituent, one or more non-hydrogen substituents independently selected from C₁₋₆ alkyl, C₁₋₆ alkoxy, halogen, or methylenedioxy.

5. The method of claim 1, further comprising reacting the compounds of Formula 1 and Formula 3 in the presence of a base selected from CsF, KF, Li₂CO₃, Na₂CO₃, NaHCO₃, K₂CO₃, Cs₂CO₃, K₂HPO₄, K₃PO₄, NaOAc, NH₄OAc, or alkyl₄NF.

6. A method of making a compound of Formula 4, 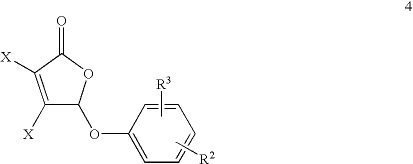
wherein X is halogen, and R² and R³ are independently H, C-6 alkyl, C₁₋₆ alkoxy, halogen, or together form a methylenedioxy, the method comprising:
reacting a compound of Formula 1, 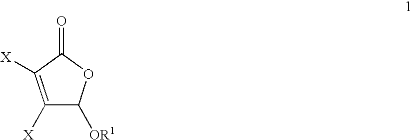
with a compound of Formula 5, 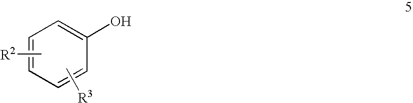
in the presence of a base and a solvent and in the absence of Pd, wherein X, R², and R³ are as defined above for Formula 4, and R¹ is C₁₋₆ alkoxycarbonyl or C₁₋₆ alkanoyl.

7. The method of claim 6, further comprising reacting the compounds of Formula 1 and Formula 5 in the presence of a base selected from CsF, KF, Li₂CO₃, Na₂CO₃, NaHCO₃, K₂CO₃, Cs₂CO₃, K₂HPO₄, K₃PO₄, NaOAc, NH₄OAc, or alkyl₄NF.

8. The method of claim 6, further comprising reacting the compounds of Formula 1 and Formula 5 in the presence of an aprotic solvent.

9. A method of making a compound of Formula 6, 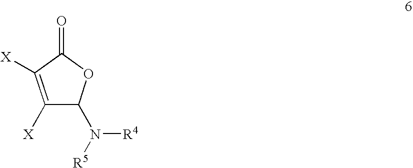
wherein X is halogen, and R⁴ and R⁵ are independently H, C₁₋₆ alkyl, C₁₋₆ alkenyl, aryl, and arylalkyl, the method comprising:
reacting a compound of Formula 1, 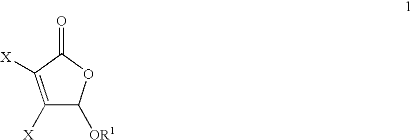
with a compound of Formula 7, 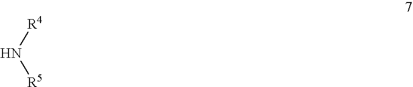
in the presence of a solvent and in the absence of Pd and other transition metals, wherein X, R⁴, and R⁵ are as defined above in Formula 6, and R¹ is C₁₋₆ alkoxycarbonyl or C₁₋₆ alkanoyl.

10. The method of claim 9, further comprising reacting the compounds of Formula 1 and Formula 7 in the presence of an aprotic solvent.

11. A compound of Formula 2, 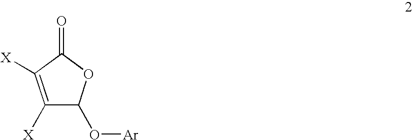
in which X is halogen and Ar is aryl.

12. The compound of claim 11, wherein Ar is a phenyl group optionally substituted with one or more non-hydrogen substituents independently selected from C₁₋₆ alkyl, C₁₋₆ alkoxy, halogen, or methylenedioxy.

13. A compound of Formula 6, 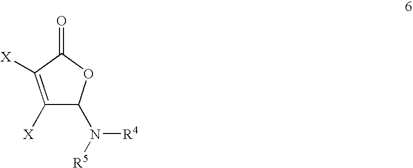
in which X is halogen, and R⁴ and R⁵ are independently H, C₁₋₆ alkyl, C₁₋₆ alkenyl, aryl, and arylalkyl.

14. A compound selected from:
4-methoxycarbonyloxy-2,3-dibromo-2-buten-4-olide;
4-t-butyloxycarbonyloxy-2,3-dichloro-2-buten-4-olide;
2,3-dichloro-4-m-tolyloxy-2-buten-4-olide;
2,3-dichloro-4-(4-methoxy-phenoxy)-2-buten-4-olide;
4-(benzo[1,3]dioxol-5-yloxy)-2,3-dichloro-2-buten-4-olide;
2,3-dichloro-4-phenoxy-2-buten-4-olide;
2,3-dichloro-4-(3-fluoro-phenoxy)-2-buten-4-olide;
2,3-dichloro-4-(3,5-dichloro-phenoxy)-2-buten-4-olide;
4-(3-bromo-phenoxy)-2,3-dichloro-2-buten-4-olide;
2,3-dibromo-4-m-tolyloxy-2-buten-4-olide;
4-benzylamino-2,3-dichloro-2-buten-4-olide;
4-all ylamino-2,3-dichloro-2-buten-4-olide;
2,3-dichloro-4-dipropylamino-2-butene-4-olide;
4-benzylamino-2,3-dibromo-2-butene-4-olide;
4-methoxycarbonyloxy-3-anilino-2-bromo-2-buten-4-olide;
4-methoxycarbonyloxy-2-chloro-3-(pyridin-3-yloxy)-2-buten-4-olide;
3-chloro-4H,9aH-1,9-dioxa-4-aza-cyclopenta[b]naphthalen-2-one;
4-methoxycarbonyloxy-2-chloro-3-m-tolylsulfanyl-2-buten-4-olide; and
2,3-dichloro-4-(2-methyl-allyl)-2-buten-4-olide.
